(19) United States
(12) Patent Application Publication
(10) Pub. No.: US 2008/0033020 A1
(43) Pub. Date: Feb. 7, 2008

(54) 3-Pyridinylethylbenzamide Derivatives as Fungicides

(76) Inventors: Darren Mansfield, Lyon (FR); Pierre-Yves Coqueron, Lyon (FR); Heiko Rieck, Sainte-Foy-Les-Lyon (FR); Philippe Desbordes, Lyon (FR); Marie-Claire Grosjean-Cournoyer, Curis Au Mont D'Or (FR)

Correspondence Address: OSTROLENK FABER GERB & SOFFEN, 1180 AVENUE OF THE AMERICAS, NEW YORK, NY, 100368403, US

(21) Appl. No.: 11/572,389

(22) PCT Filed: Jul. 21, 2005
(86) PCT No.: PCT/EP05/09181

§ 371 Date: Feb. 9, 2007

(30) Foreign Application Priority Data
Jul. 23, 2004 (EP) 04356137.2

(51) Int. Cl. A01N 43/40; C07D 213/40; C07D 213/61

(52) U.S. Cl. 514/357; 546/329; 546/330; 546/337

(57) ABSTRACT

Compound of general formula (I): 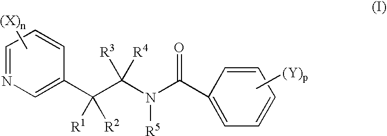 Process for preparing this compound.
Intermediate of general formula (II) to prepare compound of formula (I) 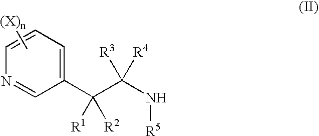
Intermediate of general formula (V) to prepare compound of formula (I): 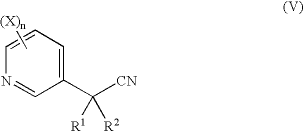 Fungicidal composition comprising a compound of general formula (I). Method for treating plants by applying a compound of general formula (I) or a composition comprising it.

The present invention relates to novel N-[2-(3-pyridinyl)ethyl]benzamide derivatives, their process of preparation, their use as fungicides, particularly in the form of fungicidal compositions, and methods for the control of phytopathogenic fungi of plants using these compounds or their compositions.

The international patent application WO 04/16088 discloses 2-pyridylethylbenzamide derivatives in which the 2-pyridyl group is substituted by at least one halogenoalkyl group and their use as fungicidal compounds. N-[2-(3-pyridinyl)ethyl]benzamide derivatives are not disclosed.

International patent application WO 01/11965 discloses a broad family of fungicidal compounds. N-[2-(3-pyridinyl)ethyl]carboxamide derivatives are not disclosed in that patent application.

It is always of high-interest in agriculture to use novel pesticidal compounds in order to avoid or to fight the development of resistant strains to the active ingredients used by the farmer. It is also of high-interest to use novel compounds being more active than those already known, with the aim of decreasing the amounts of active material to be used by the farmer, whilst at the same time maintaining an effectiveness at least equivalent to the already known compounds.

We have now found a new family of compounds which possess the above mentioned characteristics.

Accordingly, the present invention relates to N-[2-(3-pyridinyl)ethyl]benzamide derivative of general formula (I): 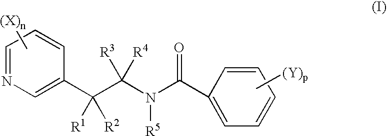
in which:
n is 1, 2, 3 or 4;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₉-alkylcarbamoyl, a (N—C₁-C₈-alkyl)oxycarbamoyl, a C₁-C₈-alkoxycarbamoyl, a (N—C₁-C₈-alkyl)-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
R¹ and R² are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkyl-sulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group; or R¹ and R² may together form a 3-, 4-, 5- or 6-membered carbocycle;
R³ and R⁴ are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkyl-sulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group; or R³ and R⁴ may together form a 3-, 4-, 5- or 6-membered carbocycle;
R⁵ is a hydrogen atom or a C₃-C₇-cycloalkyl;
p is 1, 2, 3, 4 or 5; and
Y is the same or different and is a hydrogen atom, a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a C₁-C₈-alkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₉-halogenoalkoxy having 1 to 5 halogen atoms, a C₁-C₈-alkoxy-C₂-C₈-alkenyl, a C₁-C₉-alkylsulfanyl, a C₁-C₈-halogenoalkylsulfanyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkyl-sulphonyl, a C₁-C₈-halogenoalkylsulphonyl having 1 to 5 halogen atoms or a C₁-C₈-alkylsulfonamide;

as well as its salts, N-oxydes, metallic complexes, metalloidic complexes and optically active isomers.

In the context of the present invention:

halogen means fluorine, bromine, chlorine or iodine.

carboxy means —C(═O)OH; carbonyl means —C(═O)—; carbamoyl means —C(═O)NH₂; N-hydroxycarbamoyl means —C(═O)NHOH;

an alkyl group, an alkenyl group, and an alkynyl group as well as moieties containing these terms, can be linear or branched.

In the context of the present invention, it has also to be understood that in the case of di-substituted amino and of di-substituted carbamoyl radicals, the two substituents may form together with the nitrogen atom bearing them a saturated heterocyclic ring containing 3 to 7 atoms.

Any of the compounds of the present invention can exist in one or more optical or chiral isomer forms depending on the number of asymmetric centres in the compound. The invention thus relates equally to all the optical isomers and to their racemic or scalemic mixtures (the term “scalemic” denotes a mixture of enantiomers in different proportions), and to the mixtures of all the possible stereoisomers, in all proportions. The diastereoisomers and/or the optical isomers can be separated according to the methods which are known per se by the man ordinary skilled in the art.

Any of the compounds of the present invention can also exist in one or more geometric isomer forms depending on the number of double bonds in the compound. The invention thus relates equally to all geometric isomers and to all possible mixtures, in all proportions. The geometric isomers can be separated according to general methods, which are known per se by the man ordinary skilled in the art.

Any of the compounds of general formula (I) wherein X represents a hydroxy, a sulfanyl group or an amino group may be found in its tautomeric form resulting from the shift of the proton of said hydroxy, sulfanyl or amino group. Such tautomeric forms of such compounds are also part of the present invention. More generally speaking, all tautomeric forms of compounds of general formula (I) wherein X represents a hydroxy, a sulfanyl group or an amino group, as well as the tautomeric forms of the compounds which can optionally be used as intermediates in the preparation processes, and which will be defined in the description of these processes, are also part of the present invention.

According to the present invention, the 3-pyridyl may be substituted in any position by (X)ₙ, in which X and n are as defined above. Preferably, the present invention relates to N-[2-(3-pyridinyl)ethyl]benzamide derivative of general formula (I) in which the different characteristics may be chosen alone or in combination as being:

as regards n, n is 1 or 2;

as regards X, X is chosen as being a halogen atom, a cyano group, a C₁-C₈-halogenoalkyl having 1 to 5 halogen atoms, a (C₁-C₆-alkoxyimino)-C₁-C₆-alkyl, a C₁-C₆-alkoxyimino or a C₁-C₈-alkyl.

According to the present invention, the phenyl may be substituted in any position by (Y)ₚ, in which Y and p are as defined above. Preferably, the present invention relates to N-[2-(3-pyridinyl)ethyl]benzamide derivative of general formula (I) in which the different characteristics may be chosen alone or in combination as being:

as regards p, p is 1 or 2. More preferably p is 1.

as regards Y, Y is chosen as being a halogen atom, a C₁-C₈-alkyl or a C₁-C₈-halogenoalkyl having 1 to 5 halogen atoms;

as regards the positions in which the phenyl moiety is substituted by Y, the phenyl moiety is substituted by Y in ortho position.

According to the present invention, the carbon atoms of the carboxamide moiety of the compound of formula (I) are substituted by R¹, R², R³ and R⁴, R¹, R², R³ and R⁴ being as defined above. Preferably, the present invention also relates to N-[2-(3-pyridinyl)ethyl]carboxamide derivative of general formula (I) in which the different characteristics may be chosen alone or in combination as being:

as regards R¹ and R², R¹ and R² are chosen, independently of each other, as being a hydrogen atom, a C₁-C₆-alkyl or a C₁-C₆-halogenoalkyl having 1 to 5 halogen atoms;

as regards R³ and R⁴, R³ and R⁴ are chosen, independently of each other, as being a hydrogen atom, a C₁-C₆-alkyl or a C₁-C₆-halogenoalkyl having 1 to 5 halogen atoms.

According to the present invention, the nitrogen atom of the carboxamide moiety of the compound of formula (I) is substituted by R⁵, R⁵ being a hydrogen atom or a C₃-C₇-cycloalkyl. Preferably, the C₃-C₇-cycloalkyl is cyclopropyl.

The present invention also relates to a process for the preparation of the compound of general formula (I). Thus, according to a further aspect of the present invention there is provided a process (A) for the preparation of compound of general formula (I) as defined above, which comprises reacting a 3-pyridine derivative of general formula (II) or one of its salts:

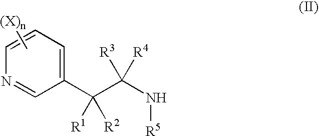

in which X, n, R¹, R², R³, R⁴ and R⁵ are as defined above;
with a carboxylic acid derivative of the general formula (III)

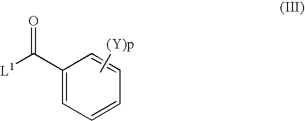

in which L¹ is a leaving group chosen as being a halogen atom, a hydroxyl group, —OR⁶, —OCOR⁶, R⁶ being a C₁-C₆ alkyl, a C₁-C₆ haloalkyl, a benzyl, 4-methoxybenzyl, pentafluorophenyl or a group of formula 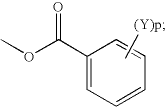
in the presence of a catalyst and, if L¹ is a hydroxyl group, in the presence of a condensing agent.

The process (A) according to the present invention is conducted in the presence of a catalyst. Suitable catalyst may be chosen as being 4-dimethyl-aminopyridine, 1-hydroxy-benzotriazole or dimethylformamide.

In case L¹ is a hydroxy group, the process according to the present invention is conducted in the presence of condensing agent. Suitable condensing agent may be chosen as being acid halide former, such as phosgene, phosphorous tribromide, phosphorous trichloride, phosphorous pentachloride, phosphorous trichloride oxide or thionyl chloride; anhydride former, such as ethyl chloroformate, methyl chloroformate, isopropyl chloroformate, isobutyl chloroformate or methanesulfonyl-chloride; carbodiimides, such as N,N′-dicyclohexylcarbodiimide (DCC) or other customary condensing agents, such as phosphorous pentoxide, polyphosphoric acid, N,N′-carbonyl-diimidazole, 2-ethoxy-N-ethoxycarbonyl-1,2-dihydroquinoline (EEDQ), triphenylphosphine/tetrachloromethane, 4-(4,6-dimethoxy[1.3.5]triazin-2-yl)-4-methylmorpholinium chloride hydrate or bromo-tripyrrolidino-phosphonium-hexafluorophosphate.

When R⁵ is a hydrogen atom, the above mentioned process (A) for the preparation of compound of general formula (I) may optionally be completed by a further step according to the following reaction scheme:

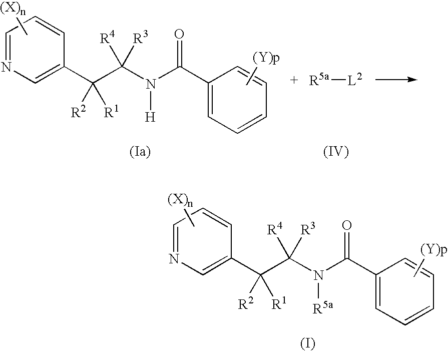

in which: R¹, R², R³, R⁴, X and n are as defined above;
R^{5a} is a C₃-C₇-cycloalkyl; and
L² is a leaving group chosen as being a halogen atom, a 4-methyl phenylsulfonyloxy or a methylsulfonyloxy;
   comprising the reaction of a compound of general formula (Ia) with a compound of general formula (IV) to provide a compound of general formula (I).

Depending on the definition of R¹, R², R³, R⁴ or R⁵, amine derivatives of general formula (II) may be prepared by different processes. One example (a) of such a process may be when:
R¹, R², X, n are as defined above;
R³, R⁴, R⁵ are hydrogen atoms; then, the amine derivative of general formula (II) may be prepared according to a process which comprises:

a first step according to reaction scheme a-1:

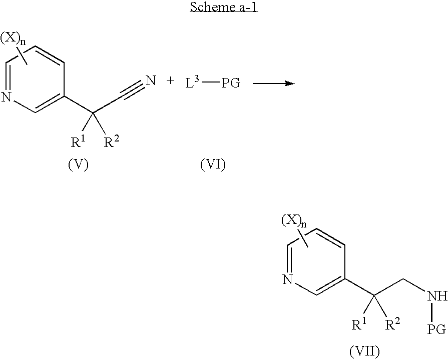

in which: R¹, R², X, n are as defined above;
L³ is a leaving group chosen as being a —OR⁶ group or a —OCOR⁶ group, R⁶ being a C₁-C₆ alkyl, a C₁-C₆ haloalkyl, a benzyl, 4-methoxybenzyl or pentafluorophenyl;
PG represents a protecting group which may be a —COOR⁶ group or —COR⁶ group, R⁶ being a C₁-C₆ alkyl, a C₁-C₆ haloalkyl, a benzyl, 4-methoxybenzyl or pentafluorophenyl;
   comprising the reduction, by hydrogenation or by an hydride donor, of a compound of general formula (V), in the presence of a catalyst and in the presence of a compound of general formula (VI) to produce a compound of general formula (VII), at a temperature of from 0° C. to 150° C. and under a pressure of from 1 bar and 100 bar;

a second step according to reaction scheme a-2:

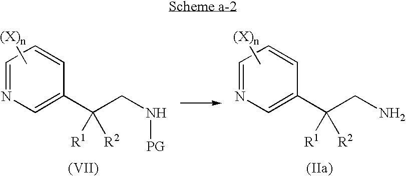

in which: R¹, R², X, n are as defined above;
PG represents a protecting group which may be a —COOR⁶ group or —COR⁶ group, R⁶ being a C₁-C₆ alkyl, a C₁-C₆ haloalkyl, a benzyl, 4-methoxybenzyl or pentafluorophenyl;
   comprising a deprotection reaction, in an acidic or in a basic medium, of a compound of general formula (VII) to provide an amine derivative of general formula (Ia) or one of its salt;

The first step (step a-1) of the process (a) is conducted in the presence of a hydride donor. Preferably, the hydride donor is chosen as being metal or metalloid hydrides such as LiAlH₄, NaBH₄, KBH₄, B₂H₆.

The first step (step a-1) of the process (a) is conducted in the presence of a catalyst. Preferably, the catalyst is chosen as being Co(II)-Chloride, Ni(II)-chloride, ammonia or one of its salt, Palladium on charcoal, Raney Nickel, Raney Cobalt or Platinum.

The first step (step a-1) of the process (a) is conducted at a temperature of from 0° C. to 150° C. Preferably the temperature is of from 10° C. to 120° C. More preferably, the temperature is of from 10° C. to 80° C.

The first step (step a-1) of the process (a) is conducted under a pressure of from 1 bar to 100 bar. Preferably the pressure is of from 1 bar to 50 bar.

The first step (step a-1) of the process (a) may be conducted in the presence of an organic solvent, of water or of a mixture thereof. Preferably, the solvent is chosen as being ether, alcohol, carboxylic acid, or a mixture thereof with water or pure water.

The present invention also relates to another process for the preparation of the compound of general formula (I). Thus, according to a further aspect of the present invention there is provided a second process (B) for the preparation compound of general formula (Ia)

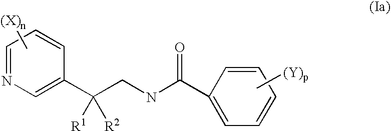

wherein R¹, R², X, Y, n and p are as defined above;
according to the following reaction scheme:

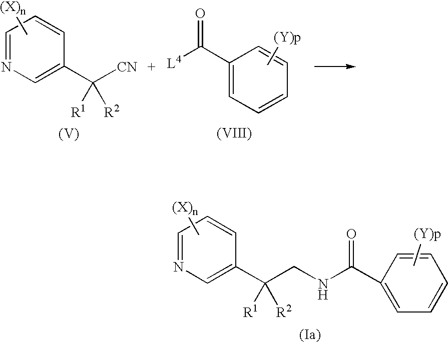

in which: R¹, R², X, Y, n and p are as defined above;
L⁴ is a leaving group chosen as being —OCOR⁶, R⁶ being a C₁-C₆ alkyl, a C₁-C₆ haloalkyl, a benzyl, 4-methoxybenzyl or pentafluorophenyl; —OCHO, —SCSN(Me)₂ or a group of formula 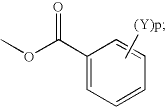
   comprising the reduction by hydrogenation or by an hydride of a compound of general formula (V) in the presence of a catalyst and in the presence of a compound of general formula (VIII) to produce a compound of general formula (Ia), at a temperature of from 0° C. to 150° C. and under a pressure of from 1 bar and 100 bar.

The process (B) according to the present invention is conducted in the presence of a hydride donor. Preferably, the hydride donor is chosen as being metal or metalloid hydrides such as LiAlH₄, NaBH₄, KBH₄, B₂H₆.

The process (B) according to the present invention is conducted in the presence of a catalyst. Preferably, the catalyst is chosen as being Co(II)-Chloride, Ni(II)-chloride, ammonia or one of its salt, Palladium on charcoal, Raney Nickel, Raney Cobalt or Platinum.

The process (B) according to the present invention is conducted at a temperature of from 0° C. to 150° C. Preferably the temperature is of from 10° C. to 120° C. More preferably, the temperature is of from 110° C. to 80° C.

The process (B) according to the present invention is conducted under a pressure of from 1 bar to 100 bar. Preferably the pressure is of from 1 bar to 50 bar.

The process (B) according to the present invention may be conducted in the presence of an organic solvent, of water or of a mixture thereof. Preferably, the solvent is chosen as being ether, alcohol, carboxylic acid, or a mixture thereof with water or pure water.

The compound according to the present invention can be prepared according to the general processes of preparation described above. It will nevertheless be understood that, on the basis of his general knowledge and of available publications, the skilled worker will be able to adapt this method according to the specifics of each of the compounds, which it is desired to synthesise.

On the basis of his general knowledge and of available publications, the skilled worker will also be able to prepare intermediate compound of formula (V) according to the present invention.

The compound of general formula (II) used as an intermediate for the preparation of compound of general formula (I) is novel. Therefore, the present invention also relates to novel intermediate compound useful for the preparation of compound of general formula (I). Thus, according to the present invention, there is provided a compound of general formula (II): 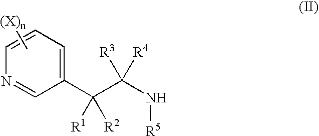
in which X, n, R¹, R², R³, R⁴ and R⁵ are as defined above.

The compound of general formula (V) used as an intermediate for the preparation of compound of general formula (I) is novel. Therefore, the present invention also relates to novel intermediate compound useful for the preparation of compound of general formula (I). Thus, according to the present invention, there is provided a compound of general formula (V): 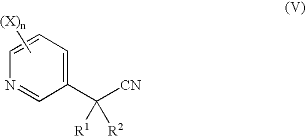
in which X, n, R¹ and R² are as defined above.

The present invention also relates to a fungicidal composition comprising an effective amount of an active material of general formula (I). Thus, according to the present invention, there is provided a fungicidal composition comprising, as an active ingredient, an effective amount of a compound of general formula (I) as defined above and an agriculturally acceptable support, carrier or filler.

In the present specification, the term “support” denotes a natural or synthetic, organic or inorganic material with which the active material is combined to make it easier to apply, notably to the parts of the plant. This support is thus generally inert and should be agriculturally acceptable. The support may be a solid or a liquid. Examples of suitable supports include clays, natural or synthetic silicates, silica, resins, waxes, solid fertilisers, water, alcohols, in particular butanol, organic solvents, mineral and plant oils and derivatives thereof. Mixtures of such supports may also be used.

The composition may also comprise additional components. In particular, the composition may further comprise a surfactant. The surfactant can be an emulsifier, a dispersing agent or a wetting agent of ionic or non-ionic type or a mixture of such surfactants. Mention may be made, for example, of polyacrylic acid salts, lignosulphonic acid salts, phenolsulphonic or naphthalenesulphonic acid salts, polycondensates of ethylene oxide with fatty alcohols or with fatty acids or with fatty amines, substituted phenols (in particular alkylphenols or arylphenols), salts of sulphosuccinic acid esters, taurine derivatives (in particular alkyl taurates), phosphoric esters of polyoxyethylated alcohols or phenols, fatty acid esters of polyols, and derivatives of the above compounds containing sulphate, sulphonate and phosphate functions. The presence of at least one surfactant is generally essential when the active material and/or the inert support are water-insoluble and when the vector agent for the application is water. Preferably, surfactant content may be comprised between 5% and 40% by weight of the composition.

Optionally, additional components may also be included, e.g. protective colloids, adhesives, thickeners, thixotropic agents, penetration agents, stabilisers, sequestering agents. More generally, the active materials can be combined with any solid or liquid additive, which complies with the usual formulation techniques.

In general, the composition according to the invention may contain from 0.05 to 99% (by weight) of active material, preferably 10 to 70% by weight.

Compositions according to the present invention can be used in various forms such as aerosol dispenser, capsule suspension, cold fogging concentrate, dustable powder, emulsifiable concentrate, emulsion oil in water, emulsion water in oil, encapsulated granule, fine granule, flowable concentrate for seed treatment, gas (under pressure), gas generating product, granule, hot fogging concentrate, macrogranule, microgranule, oil dispersible powder, oil miscible flowable concentrate, oil miscible liquid, paste, plant rodlet, powder for dry seed treatment, seed coated with a pesticide, soluble concentrate, soluble powder, solution for seed treatment, suspension concentrate (flowable concentrate), ultra low volume (ulv) liquid, ultra low volume (ulv) suspension, water dispersible granules or tablets, water dispersible powder for slurry treatment, water soluble granules or tablets, water soluble powder for seed treatment and wettable powder.

These compositions include not only compositions which are ready to be applied to the plant or seed to be treated by means of a suitable device, such as a spraying or dusting device, but also concentrated commercial compositions which must be diluted before application to the crop.

The compounds of the invention can also be mixed with one or more insecticides, fungicides, bactericides, attractant acaricides or pheromones or other compounds with biological activity. The mixtures thus obtained have a broadened spectrum of activity. The mixtures with other fungicides are particularly advantageous. Examples of suitable fungicide mixing partners may be selected in the following lists:

B1) a compound capable to inhibit the nucleic acid synthesis like benalaxyl, benalaxyl-M, bupirimate, chiralaxyl, clozylacon, dimethirimol, ethirimol, furalaxyl, hymexazol, metalaxyl-M, ofurace, oxadixyl, oxolinic acid;

B2) a compound capable to inhibit the mitosis and cell division like benomyl, carbendazim, diethofencarb, fuberidazole, pencycuron, thiabendazole thiophanate-methyl, zoxamide;

B3) a compound capable to inhibit the respiration for example

as CI-respiration inhibitor like diflumetorim;

as CII-respiration inhibitor like boscalid, carboxin, fenfuram, flutolanil, furametpyr, mepronil, oxycarboxine, penthiopyrad, thifluzamide;

as CIII-respiration inhibitor like azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin;

B4) a compound capable of to act as an uncoupler like dinocap, fluazinam;

B5) a compound capable to inhibit ATP production like fentin acetate, fentin chloride, fentin hydroxide, silthiofam;

B6) a compound capable to inhibit AA and protein biosynthesis like andoprim, blasticidin-S, cyprodinil, kasugamycin, kasugamycin hydrochloride hydrate, mepanipyrim, pyrimethanil;

B7) a compound capable to inhibit the signal transduction like fenpiclonil, fludioxonil, quinoxyfen;

B8) a compound capable to inhibit lipid and membrane synthesis like chlozolinate, iprodione, procymidone, vinclozolin, pyrazophos, edifenphos, iprobenfos (IBP), isoprothiolane, tolclofos-methyl, biphenyl, iodocarb, propamocarb, propamocarb-hydrochloride;

B9) a compound capable to inhibit ergosterol biosynthesis like fenhexamid, azaconazole, bitertanol, bromuconazole, cyproconazole, diclobutrazole, difenoconazole, diniconazole, diniconazole-M, epoxiconazole, etaconazole, fenbuconazole, fluquinconazole, flusilazole, flutriafol, furconazole, furconazole-cis, hexaconazole, imibenconazole, ipconazole, metconazole, myclobutanil, paclobutrazol, penconazole, propiconazole, prothioconazole, simeconazole, tebuconazole, tetraconazole, triadimefon, triadimenol, triticonazole, uniconazole, voriconazole, imazalil, imazalil sulfate, oxpoconazole, fenarimol, flurprimidol, nuarimol, pyrifenox, triforine, pefurazoate, prochloraz, triflumizole, viniconazole, aldimorph, dodemorph, dodemorph acetate, fenpropimorph, tridemorph, fenpropidin, spiroxamine, naftifine, pyributicarb, terbinafine;

B10) a compound capable to inhibit cell wall synthesis like benthiavalicarb, bialaphos, dimethomorph, flumorph, iprovalicarb, polyoxins, polyoxorim, validamycin A;

B11) a compound capable to inhibit melanine biosynthesis like carpropamid, diclocymet, fenoxanil, phtalide, pyroquilon, tricyclazole;

B12) a compound capable to induce a host defence like acibenzolar-S-methyl, probenazole, tiadinil;

B13) a compound capable to have a multisite action like captafol, captan, chlorothalonil, copper preparations such as copper hydroxide, copper naphthenate, copper oxychloride, copper sulphate, copper oxide, oxine-copper and Bordeaux mixture, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, propineb, sulphur and sulphur preparations including calcium polysulphide, thiram, tolylfluanid, zineb, ziram;

B14) a compound selected in the following list: amibromdole, benthiazole, bethoxazin, capsimycin, carvone, chinomethionat, chloropicrin, cufraneb, cyflufenamid, cymoxanil, dazomet, debacarb, diclomezine, dichlorophen, dicloran, difenzoquat, difenzoquat methylsulphate, diphenylamine, ethaboxam, ferimzone, flumetover, flusulfamide, fosetyl-aluminium, fosetyl-calcium, fosetyl-sodium, fluopicolide, fluoroimide, hexachlorobenzene, 8-hydroxyquinoline sulfate, irumamycin, methasulphocarb, metrafenone, methyl isothiocyanate, mildiomycin, natamycin, nickel dimethyldithiocarbamate, nitrothal-isopropyl, octhilinone, oxamocarb, oxyfenthiin, pentachlorophenol and salts, 2-phenylphenol and salts, phosphorous acid and its salts, piperalin, propanosine-sodium, proquinazid, pyrrolnitrine, quintozene, tecloftalam, tecnazene, triazoxide, trichiamide, zarilamid and 2,3,5,6-tetrachloro-4-(methylsulfonyl)-pyridine, N-(4-Chloro-2-nitrophenyl)-N-ethyl-4-methyl-benzenesulfonamide, 2-amino-4-methyl-N-phenyl-5-thiazolecarboxamide, 2-chloro-N-(2,3-dihydro-1,1,3-trimethyl-1H-inden-4-yl)-3-pyridincarboxamide, 3-[5-(4-chlorophenyl)-2,3-dimethylisoxazolidin-3-yl]pyridine, cis-1-(4-chlorophenyl)-2-(1H-1,2,4-triazole-1-yl)-cycloheptanol, methyl 1-(2,3-dihydro-2,2-dimethyl-1H-inden-1-yl)-1H-imidazole-5-carboxylate, 3,4,5-trichloro-2,6-pyridinedicarbonitrile, Methyl 2-[[[cyclopropyl[(4-methoxyphenyl)imino]methyl]thio]methyl]-.alpha.-(methoxymethylene)-benzeneacetate, 4-Chloro-alpha-propynyloxy-N-[2-[3-methoxy-4-(2-propynyloxy)phenyl]ethyl]-benzeneacetamide, (2S)—N-[2-[4-[[3-(4-chlorophenyl)-2-propynyl]oxy]-3-methoxyphenyl]ethyl]-3-methyl-2-[(methylsulfonyl)amino]-butanamide, 5-chloro-7-(4-methylpiperidin-1-yl)-6-(2,4,6-trifluorophenyl)[1,2,4]triazolo[1,5-a]pyrimidine, 5-chloro-6-(2,4,6-trifluorophenyl)-N-[(1R)-1,2,2-trimethylpropyl][1,2,4]triazolo[1,5-a]pyrimidin-7-amine, 5-chloro-N-[(1R)-1,2-dimethylpropyl]-6-(2,4,6-trifluorophenyl)([1,2,4]triazolo[1,5-a]pyrimidin-7-amine, N-[1-(5-bromo-3-chloropyridin-2-yl)ethyl]-2,4-dichloronicotinamide, N-(5-bromo-3-chloropyridin-2-yl)methyl-2,4-dichloronicotinamide, 2-butoxy-6-iodo-3-propyl-benzopyranon-4-one, N-{(Z)-[(cyclopropylmethoxy)imino][6-(difluoromethoxy)-2,3-difluorophenyl]methyl}-2-phenylacetamide, N-(3-ethyl-3,5,5-trimethyl-cyclohexyl)-3-formylamino-2-hydroxy-benzamide, 2-[[[[1-[3(1 Fluoro-2-phenylethyl)oxy]phenyl]ethylidenelamino]oxy]methyl]-alpha-(methoxyimino)-N-methyl-alphaE-benzeneacetamide, N-{2-[3-chloro-5-(trifluoromethyl)pyridin-2-yl]ethyl}-2-(trifluoromethyl)benzamide, N-(3′,4′-dichloro-5-fluorobiphenyl-2-yl)-3-(difluoromethyl)-1-methyl-1H-pyrazole-4-carboxamide, 2-(2-{[6-(3-chloro-2-methylphenoxy)-5-fluoropyrimidin-4-yl]oxy}phenyl)-2-(methoxyimino)-N-methylacetamide, 1-[(4-methoxyphenoxy)methyl]-2,2-dimethylpropyl-1H-imidazole-1-carboxylic acid, O-[1-[(4-methoxyphenoxy)methyl]-2,2-dimethylpropyl]-1H-imidazole-1-carbothioic acid.

The composition according to the invention comprising a mixture of a compound of formula (I) with a bactericide compound may also be particularly advantageous. Examples of suitable bactericide mixing partners may be selected in the following list: bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

The fungicidal compositions of the present invention can be used to curatively or preventively control the phytopathogenic fungi of crops. Thus, according to a further aspect of the present invention, there is provided a method for curatively or preventively controlling the phytopathogenic fungi of crops characterised in that a fungicidal composition as hereinbefore defined is applied to the seed, the plant and/or to the fruit of the plant or to the soil in which the plant is growing or in which it is desired to grow.

The composition as used against phytopathogenic fungi of crops comprises an effective and non-phytotoxic amount of an active material of general formula (I).

The expression “effective and non-phytotoxic amount” means an amount of composition according to the invention which is sufficient to control or destroy the fungi present or liable to appear on the crops, and which does not entail any appreciable symptom of phytotoxicity for the said crops. Such an amount can vary within a wide range depending on the fungus to be controlled, the type of crop, the climatic conditions and the compounds included in the fungicidal composition according to the invention.

This amount can be determined by systematic field trials, which are within the capabilities of a person skilled in the art.

The method of treatment according to the present invention is useful to treat propagation material such as tubers or rhizomes, but also seeds, seedlings or seedlings pricking out and plants or plants pricking out. This method of treatment can also be useful to treat roots. The method of treatment according to the present invention can also be useful to treat the overground parts of the plant such as trunks, stems or stalks, leaves, flowers and fruits of the concerned plant.

Among the plants that can be protected by the method according to the present invention, mention may be made of cotton; flax; vine; fruit or vegetable crops such as Rosaceae sp. (for instance pip fruit such as apples and pears, but also stone fruit such as apricots, almonds and peaches), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for instance banana trees and plantins), Rubiaceae sp., Theaceae sp., Sterculiceae sp., Rutaceae sp. (for instance lemons, oranges and grapefruit); Solanaceae sp. (for instance tomatoes), Liliaceae sp., Asteraceae sp. (for instance lettuces), Umbelliferae sp., Cruciferae sp., Chenopodiaceae sp., Cucurbitaceae sp., Papilionaceae sp. (for instance peas), Rosaceae sp. (for instance strawberries); major crops such as Graminae sp. (for instance maize, lawn or cereals such as wheat, rice, barley and triticale), Asteraceae sp. (for instance sunflower), Cruciferae sp. (for instance colza), Fabacae sp. (for instance peanuts), Papilionaceae sp. (for instance soybean), Solanaceae sp. (for instance potatoes), Chenopodiaceae sp. (for instance beetroots); horticultural and forest crops; as well as genetically modified homologues of these crops.

Among the diseases of plants or crops that can be controlled by the method according to the present invention, mention may be made of:

Powdery mildew diseases such as:

Blumeria diseases, caused for example by Blumeria graminis;

Podosphaera diseases, caused for example by Podosphaera leucotricha;

Sphaerotheca diseases, caused for example by Sphaerotheca fuliginea;

Uncinula diseases, caused for example by Uncinula necator;

Rust diseases such as:

Gymnosporangium diseases, caused for example by Gymnosporangium sabinae;

Hemileia diseases, caused for example by Hemileia vastatrix;

Phakopsora diseases, caused for example by Phakopsora pachyrhizi or Phakopsora meibomiae;

Puccinia diseases, caused for example by Puccinia recondita;

Uromyces diseases, caused for example by Uromyces appendiculatus;

Oomycete diseases such as:

Bremia diseases, caused for example by Bremia lactucae;

Peronospora diseases, caused for example by Peronospora pisi or P. brassicae;

Phytophthora diseases, caused for example by Phytophthora infestans;

Plasmopara diseases, caused for example by Plasmopara viticola;

Pseudoperonospora diseases, caused for example by Pseudoperonospora humuli or

Pseudoperonospora cubensis;

Pythium diseases, caused for example by Pythium ultimum;

Leafspot, leaf blotch and leaf blight diseases such as:

Alternaria diseases, caused for example by Alternaria solani;

Cercospora diseases, caused for example by Cercospora beticola;

Cladiosporum diseases, caused for example by Cladiosporium cucumerinum;

Cochliobolus diseases, caused for example by Cochliobolus sativus;

Colletotrichum diseases, caused for example by Colletotrichum lindemuthanium;

Cycloconium diseases, caused for example by Cycloconium oleaginum;

Diaporthe diseases, caused for example by Diaporthe citri;

Elsinoe diseases, caused for example by Elsinoe fawcettii;

Gloeosporium diseases, caused for example by Gloeosporium laeticolor;

Glomerella diseases, caused for example by Glomerella cingulata;

Guignardia diseases, caused for example by Guignardia bidwelli;

Leptosphaeria diseases, caused for example by Leptosphaeria maculans; Leptosphaeria nodorum;

Magnaporthe diseases, caused for example by Magnaporthe grisea;

Mycosphaerella diseases, caused for example by Mycosphaerella graminicola; Mycosphaerella arachidicola; Mycosphaerella fijiensis;

Phaeosphaeria diseases, caused for example by Phaeosphaeria nodorum;

Pyrenophora diseases, caused for example by Pyrenophora teres;

Ramularia diseases, caused for example by Ramularia collo-cygni;

Rhynchosporium diseases, caused for example by Rhynchosporium secalis;

Septoria diseases, caused for example by Septoria apii or Septoria lycopercisi;

Typhula diseases, caused for example by Typhula incarnata;

Venturia diseases, caused for example by Venturia inaequalis;

Root and stem diseases such as:

Corticium diseases, caused for example by Corticium graminearum;

Fusarium diseases, caused for example by Fusarium oxysporum;

Gaeumannomyces diseases, caused for example by Gaeumannomyces graminis;

Rhizoctonia diseases, caused for example by Rhizoctonia solani;

Tapesia diseases, caused for example by Tapesia acuformis;

Thielaviopsis diseases, caused for example by Thielaviopsis basicola;

Ear and panicle diseases such as:

Alternaria diseases, caused for example by Alternaria spp.;

Aspergillus diseases, caused for example by Aspergillus flavus;

Cladosporium diseases, caused for example by Cladosporium spp.;

Claviceps diseases, caused for example by Claviceps purpurea;

Fusarium diseases, caused for example by Fusarium culmorum;

Gibberella diseases, caused for example by Gibberella zeae;

Monographella diseases, caused for example by Monographella nivalis;

Smut and bunt diseases such as:

Sphacelotheca diseases, caused for example by Sphacelotheca reiliana;

Tilletia diseases, caused for example by Tilletia caries;

Urocystis diseases, caused for example by Urocystis occulta;

Ustilago diseases, caused for example by Ustilago nuda;

Fruit rot and mould diseases such as:

Aspergillus diseases, caused for example by Aspergillus flavus;

Botrytis diseases, caused for example by Botrytis cinerea;

Penicillium diseases, caused for example by Penicillium expansum;

Sclerotinia diseases, caused for example by Sclerotinia sclerotiorum;

Verticilium diseases, caused for example by Verticilium alboatrum;

Seed and soilborne decay, mould, wilt, rot and damping-off diseases:

Fusarium diseases, caused for example by Fusarium culmorum;

Phytophthora diseases, caused for example by Phytophthora cactorum;

Pythium diseases, caused for example by Pythium ultimum;

Rhizoctonia diseases, caused for example by Rhizoctonia solani;

Sclerotium diseases, caused for example by Sclerotium rolfsii;

Microdochium diseases, caused for example by Microdochium nivale;

Canker, broom and dieback diseases such as:

Nectria diseases, caused for example by Nectria galligena;

Blight diseases such as:

Monilinia diseases, caused for example by Monilinia laxa;

Leaf blister or leaf curl diseases such as:

Taphrina diseases, caused for example by Taphrina deformans;

Decline diseases of wooden plants such as:

Esca diseases, caused for example by Phaemoniella clamydospora;

Diseases of flowers and Seeds such as:

Botrytis diseases, caused for example by Botrytis cinerea;

Diseases of tubers such as:

Rhizoctonia diseases, caused for example by Rhizoctonia solani.

The fungicide composition according to the present invention may also be used against fungal diseases liable to grow on or inside timber. The term “timber” means all types of species of wood, and all types of working of this wood intended for construction, for example solid wood, high-density wood, laminated wood, and plywood. The method for treating timber according to the invention mainly consists in contacting one or more compounds of the present invention, or a composition according to the invention; this includes for example direct application, spraying, dipping, injection or any other suitable means.

The dose of active material usually applied in the treatment according to the present invention is generally and advantageously between 10 and 800 g/ha, preferably between 50 and 300 g/ha for applications in foliar treatment. The dose of active substance applied is generally and advantageously between 2 and 200 g per 100 kg of seed, preferably between 3 and 150 g per 100 kg of seed in the case of seed treatment. It is clearly understood that the doses indicated above are given as illustrative examples of the invention. A person skilled in the art will know how to adapt the application doses according to the nature of the crop to be treated.

The fungicidal composition according to the present invention may also be used in the treatment of genetically modified organisms with the compounds according to the invention or the agrochemical compositions according to the invention. Genetically modified plants are plants into whose genome a heterologous gene encoding a protein of interest has been stably integrated. The expression “heterologous gene encoding a protein of interest” essentially means genes which give the transformed plant new agronomic properties, or genes for improving the agronomic quality of the transformed plant.

The compositions according to the present invention may also be used for the preparation of composition useful to curatively or preventively treat human and animal fungal diseases such as, for example, mycoses, dermatoses, trichophyton diseases and candidiases or diseases caused by Aspergillus spp., for example Aspergillus fumigatus.

The aspects of the present invention will now be illustrated with reference to the following tables of compounds and examples. The following Table illustrates in a non-limiting manner examples of fungicidal compounds according to the present invention. In the following Examples, M+1 (or M−1) means the molecular ion peak, plus or minus 1 a.m.u. (atomic mass units) respectively, as observed in mass spectroscopy and M (ApcI+) means the molecular ion peak as it was found via positive atmospheric pressure chemical ionisation in mass spectroscopy.

TABLE 1

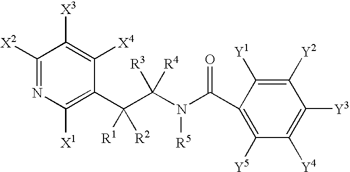

| Com- | | | | | | | | | | | | | | | |
|---|---|---|---|---|---|---|---|---|---|---|---|---|---|---|---|
| pound | | | | | | | | | | | | | | | |
| n° | X¹ | X² | X³ | X⁴ | R¹ | R² | R³ | R⁴ | R⁵ | Y¹ | Y² | Y³ | Y⁴ | Y⁵ | M + 1 |
| 1 | H | CF₃ | H | H | H | H | H | H | H | Cl | H | H | H | H | 329 |
| 2 | H | CF₃ | H | H | H | H | H | H | H | Me | H | H | H | H | 309 |
| 3 | H | CF₃ | H | H | H | H | H | H | H | Br | H | H | H | H | 373 |
| 4 | H | CF₃ | H | H | H | H | H | H | H | CF₃ | H | H | H | H | 363 |
| 5 | H | Cl | H | H | H | H | H | H | H | Cl | H | H | H | H | 295 |
| 6 | H | Cl | H | H | H | H | H | H | H | CF₃ | H | H | H | H | 329 |
| 7 | H | Cl | H | H | Me | H | H | H | H | Cl | H | H | H | H | 309 |
| 8 | H | Cl | H | H | Me | H | H | H | H | Me | H | H | H | H | 289 |
| 9 | H | Cl | H | H | Me | H | H | H | H | Br | H | H | H | H | 353 |
| 10 | H | Cl | H | H | Me | H | H | H | H | I | H | H | H | H | 401 |
| 11 | H | Cl | H | H | Me | H | H | H | H | CF₃ | H | H | H | H | 343 |
| 12 | H | Cl | H | H | H | H | H | H | H | I | H | H | H | H | 387 |
| 13 | Cl | H | H | Cl | H | H | H | H | H | CF₃ | H | H | H | H | 363 |

### EXAMPLE OF PROCESS FOR THE PREPARATION OF THE COMPOUND OF GENERAL FORMULA (I)

### Preparation of N-[2-(6-chloro-3-pyridinyl)propyl]-2-(trifluoromethyl)benzamide (Compound 11)

The 2-(2-chloro-5-pyridinyl)propionitrile (0.50 mmol, 90 mg), the 2-(trifluoromethyl)phenyl anhydride (0.10 mmol, 370 mg) and nickel chloride hexahydrate (0.50 mmol, 119 mg) are added to methanol (5 ml) at 0° C.

Sodium borohydride (3.50 mmol, 131 mg) is added portionwise to the reaction mixture.

After completion of the addition, the reaction mixture is allowed to room temperature, stirred for eighteen hours, concentrated to dryness and separated on silica (ethyl acetate/heptane=3/7) to yield to 70 mg (41%) of N-[2-(6-chloro-3-pyridinyl)propyl]-2-(trifluoromethyl)benzamide (compound II).

Mass spectrum: [M+1]=343.

### Preparation of N-[2-(6-chloro-3-pyridinyl)ethyl]-2-iodobenzamide (Compound 12)

### Preparation of tert-butyl 2-(6-chloro-3-pyridinyl)ethylcarbamate

The 2-(6-chloro-3-pyridinyl)acetonitrile (4.46 mmol, 0.68 g), the di-terbutyl carbonate (8.92 mmol, 1.95 g) and nickel chloride hexahydrate (4.46 mmol, 1.06 g) are added to methanol (30 ml) at room temperature. Sodium borohydride (13.37 mmol, 0.50 g) is added portionwise to the reaction mixture.

The reaction mixture is allowed to room temperature and stirred for three hours. After filtration on celite pad and concentration in vacuo, 50 ml of ethyl acetate are added to the crude material which is washed twice with 50 ml of water.

The organic phase is dried over magnesium sulfate, filtered and concentrated in vacuo.

The crude material is purified over silica (ethyl acetate/heptane=30/70) to yield to 0.32 g (28%) of tert-butyl 2-(6-chloro-3-pyridinyl)ethylcarbamate.

Mass spectrum: [M+1]=257.

### Preparation of 2-(6-chloro-3-pyridinyl)ethylamine

The tert-butyl 2-(6-chloro-3-pyridinyl)ethylcarbamate (1.25 mmol, 320 mg) is dissolved in 6 ml of dichloromethane.Trifluoroacetic acid (0.6 ml) is added at room temperature. After one night of stirring at room temperature, the reaction mixture is concentrated in vacuo.

The pH is adjusted to twelve with sodium hydroxide 30%, extracted thrice with 30 ml of dichloromethane.

The organic phase is dried over magnesium sulfate, filtered and concentrated to yield to 194 mg of essentially pure 2-(6-chloro-3-pyridinyl)ethylamine (99%). RMN ¹H CDCl₃: δ (ppm): 8.15 (1H, s); 7.45 (1H, d); 7.18 (1H, d); 2.85 (2H, m); 2.61 (2H, m).

### Preparation of N-[2-(6-chloro-3-pyridinyl)ethyl]-2-iodobenzamide (Compound 12)

The 2-(6-chloro-3-pyridinyl)ethylamine (0.41 mmol, 64 mg), the 2-iodobenzoyl chloride (0.82 mmol, 218 mg) are added successively to a suspension of potassium bicarbonate (1.27 mmol, 169 mg) are in acetonitrile (5 ml) at room temperature. The reaction mixture is stirred at room temperature overnight. Ethyl acetate (20 ml), water saturated with sodium bicanronate (20 ml) are added.

After separation, the organic phase is washed with mater (20 ml), dried over magnesium sulfate, filtered and concentrated to dryness to yield to, after separation on silica (ethyl acetate/heptane=3/7), 41 mg (26%) of N-[2-(6-chloro-3-pyridinyl)ethyl]-2-iodobenzamide.

Mass spectrum: [M+1]=387

### EXAMPLES OF BIOLOGICAL ACTIVITY OF THE COMPOUND OF GENERAL FORMULA (I)

### Example A

### In Vivo Test on Alternaria brassicae (Leaf Spot of Crucifers)

The active ingredient tested is prepared by potter homogenisation in a concentrated suspension type formulation at 100 g/l. This suspension is then diluted with water to obtain the desired active material concentration.

Radish plants (Pernot variety) in starter cups, sown on a 50/50 peat soil-pozzolana substrate and grown at 18-20° C., are treated at the cotyledon stage by spraying with the aqueous suspension described above.

Plants, used as controls, are treated with an aqueous solution not containing the active material.

After 24 hours, the plants are contaminated by spraying them with an aqueous suspension of Alternaria brassicae spores (40,000 spores per cm³). The spores are collected from a 12 to 13 days-old culture.

The contaminated radish plants are incubated for 6-7 days at about 18° C., under a humid atmosphere.

Grading is carried out 6 to 7 days after the contamination, in comparison with the control plants.

Under these conditions, good (at least 50%) or total protection is observed at a dose of 330 ppm or 110 ppm with the following compounds: 1, 2, 3, 4, 5, 6, 7, 8, 9, 10 and 11.

### Example B

### In Vivo Test on Pyrenophora teres (Barley Net Blotch)

The active ingredient tested is prepared by potter homogenisation in a concentrated suspension type formulation at 100 g/l. This suspension is then diluted with water to obtain the desired active material concentration.

Barley plants (Express variety) in starter cups, sown on a 50/50 peat soil-pozzolana substrate and grown at 12° C., are treated at the 1-leaf stage (10 cm tall) by spraying with the aqueous suspension described above. Plants, used as controls, are treated with an aqueous solution not containing the active material.

After 24 hours, the plants are contaminated by spraying them with an aqueous suspension of Pyrenophora teres spores (12,000 spores per ml). The spores are collected from a 12-day-old culture. The contaminated barley plants are incubated for 24 hours at about 20° C. and at 100% relative humidity, and then for 12 days at 80% relative humidity.

Grading is carried out 12 days after the contamination, in comparison with the control plants. Under these conditions, good (at least 50%) or total protection is observed at a dose of 330 ppm with the following compounds: 9, 10 and 13.

The N-{1-methylcarbamoyl-2-[3-chloro-5-(trifluoromethyl)-2-pyridinyl]-ethyl}-4-phenylbenzamide disclosed by Patent Application WO 01/11965 (see compound 316 in Table D) showed poor effectiveness on Alternaria brassicae, and zero effectiveness on Botrytis cinerea at 330 ppm; the N-{1-ethylcarbamoyl-2-[3-chloro-5-(trifluoromethyl)-2-pyridinyl]ethyl}-3-nitrobenzamide also disclosed by Patent Application WO 01/11965 (see compound 307 in Table D) showed poor effectiveness on Alternaria brassicae and zero effectiveness on Botrytis cinerea at 330 ppm; the N-{1-ethylcarbamoyl-2-[3-chloro-5-(trifluoromethyl)-2-pyridinyl]-ethyl}-benzamide and the N-{1-methylcarbamoyl-2-[3-chloro-5-(trifluoromethyl)-2-pyridinyl]ethyl}-benzamide also disclosed by Patent Application WO 01/11965 (see compounds 304 and 314 in Table D) showed zero effectiveness on Botrytis cinerea at 330 ppm; and the N-{1-ethylcarbamoyl-2-[3-chloro-5-(trifluoromethyl)-2-pyridinyl]ethyl}-4-chlorobenzamide, the N-{1-ethylcarbamoyl-2-[3-chloro-5-(trifluoromethyl)-2-pyridinyl]ethyl}-2-bromobenzamide and the N-{1-methylcarbamoyl-2-[3-chloro-5-(trifluoromethyl)-2-pyridinyl]ethyl}-4-methoxybenzamide also disclosed by Patent Application WO 01/11965 (see compounds 306, 310 and 315 in Table D) showed zero effectiveness on Botrytis cinerea at 330 ppm.

The N-{[3-chloro-5-(trifluoromethyl)-2-pyridinyl]methyl}-5-thienylacetamide disclosed by Patent Application WO 01/11965 (see compound 101 in table B) showed poor efficacy against Alternaria brassicae and no efficacy against Botrytis cinerea and Peronospora parasitica at 330 ppm.

1. A compound of general formula (I): 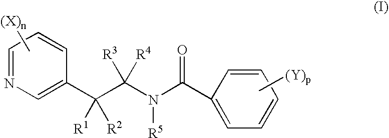
In which:
n is 1, 2, 3 or 4;
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₉-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a (N—C₁-C₈-alkyl)oxycarbamoyl, a C₁-C₈-alkoxycarbamoyl, a (N—C₁-C₈-alkyl)-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
R¹ and R² are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group;
or R¹ and R² may together form a 3-, 4-, 5- or 6-membered carbocycle;
R³ and R⁴ are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group;
or R³ and R⁴ may together form a 3-, 4-, 5- or 6-membered carbocycle;
R⁵ is a hydrogen atom or a C₃-C₇-cycloalkyl;
p is 1, 2, 3, 4 or 5; and
Y is the same or different and is a hydrogen atom, a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a C₁-C₈-alkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkoxy-C₂-C₈-alkenyl, a C₁-C₈-alkylsulfanyl, a C₁-C₈-halogenoalkylsulfanyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms or a C₁-C₈-alkylsulfonamide;
as well as its salts, N-oxydes, metallic complexes, metalloidic complexes and optically active isomers.

2. A compound according to claim 1, characterised in that n is 1 or 2.

3. A compound according to claim 1, characterised in that X is a halogen atom, a cyano group, a C₁-C₈-halogenoalkyl having 1 to 5 halogen atoms, a (C₁-C₆-alkoxyimino)-C₁-C₆-alkyl, a C₁-C₆-alkoxyimino or a C₁-C₈-alkyl.

4. A compound according to claim 1, characterised in that p is 1 or 2.

5. A compound according to claim 1, characterised in that Y is a halogen atom, a C₁-C₈-alkyl or a C₁-C₈-halogenoalkyl having 1 to 5 halogen atoms.

6. A compound according to claim 1, characterised in that phenyl moiety is substituted by Y in ortho position.

7. A compound according to claim 1, characterised in that R¹ and R² are chosen, independently of each other, as being a hydrogen atom, a C₁-C₆-alkyl or a C₁-C₆-halogenoalkyl having 1 to 5 halogen atoms;

8. A compound according to claim 1, characterised in that R³ and R⁴ are chosen, independently of each other, as being a hydrogen atom, a C₁-C₆-alkyl or a C₁-C₆-halogenoalkyl having 1 to 5 halogen atoms.

9. A process for the preparation of a compound of general formula (I) as defined in claim 1, which comprises reacting a 3-pyridine derivative of general formula (II) or one of its salt: 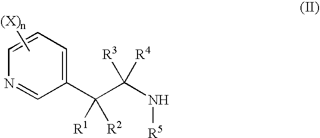
with a carboxylic acid derivative of the general formula (III) 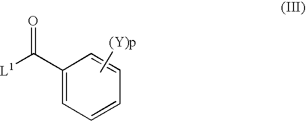
in which L¹ is a leaving group chosen as being a halogen atom, a hydroxyl group, —OR⁶, —OCOR⁶, R⁶ being a C₁-C₆ alkyl, a C₁-C₆ haloalkyl, a benzyl, 4-methoxybenzyl, pentafluorophenyl or a group of formula 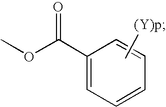
in the presence of a catalyst and, if L¹ is a hydroxyl group, in the presence of a condensing agent.

10. A process according to claim 9, characterised in that R⁵ is a hydrogen atom, and that the process is completed by a further step according to the following reaction scheme: 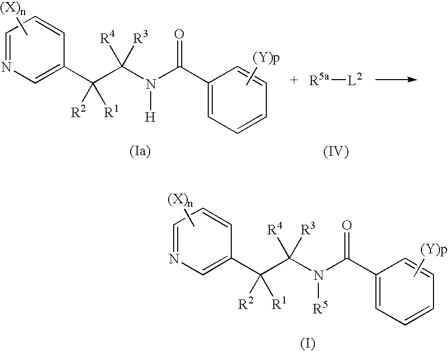
in which:
R^{5a} is a C₃-C₇-cycloalkyl; and
L² is a leaving group chosen as being a halogen atom, a 4-methyl phenylsulfonyloxy or a methylsulfonyloxy;
comprising the reaction of a compound of general formula (Ia) with a compound of general formula (IV) to provide a compound of general formula (I).

11. A process for the preparation of a compound of general formula (Ia) 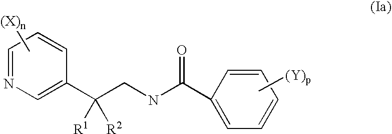
wherein
R¹ and R² are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkoxy a C₂-C₆-alkenyl a C₁-C₆-alkyl a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group;
or R¹ and R² may together form a 3-, 4-, 5- or 6-membered carbocycle,
X is the same or different and is a halogen atom, a nitro group, a cyano group, a hydroxy group, an amino group, a sulfanyl group, a pentafluoro-λ⁶-sulfanyl group a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₃-C₈-alkynyloxy, a C₁-C₈-halogenoalkynyloxy having 1 to 5 halogen atoms, a C₁-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a (N—C₁-C₈-alkyl)oxycarbamoyl, a C₁-C₈-alkoxycarbamoyl, a (N—C₁-C₈-alkyl)-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
Y is the same or different and is a hydrogen atom, a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a C₁-C₈-alkyl, a C₁-C₈-halogenoalkyl having 1 to 5 halogen atoms, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkoxy having 1 to 5 halogen atoms, a C₁-C₈-alkoxy-C₂-C₈-alkenyl, a C₁-C₈-alkylsulfanyl, a C₁-C₈-halogenoalkylsulfanyl having 1 to 5 halogen atoms, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylsulphenyl, a C₁-C₈-halogenoalkylsulphenyl having 1 to 5 halogen atoms, a C₁-C₈-alkylsulphinyl a C₁-C₈-halogenoalkylsulphinyl having 1 to 5 halogen atoms, a C₁-C₈-alkylsulphonyl a C₁-C₈-halogenoalkylsulphonyl having 1 to 5 halogen atoms or a C₁-C₈-alkylsulfonamide;
n is 1, 2, 3 or 4;
p is 1, 2, 3, 4 or 5;
according to the following reaction scheme 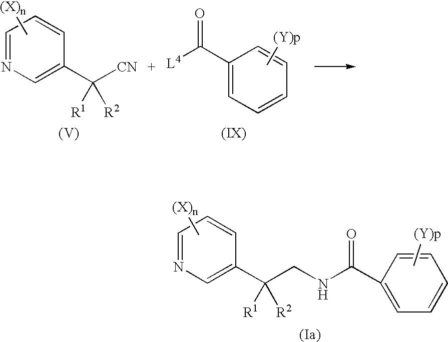
in which:
L⁴ is a leaving group chosen as being —OCOR⁶, R⁶ being a C₁-C₆ alkyl, a C₁-C₆ haloalkyl, a benzyl, 4-methoxybenzyl or pentafluorophenyl; —OCHO, —SCSN(Me)₂ or a group of formula 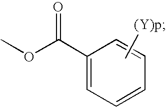
comprising the reduction by hydrogenation or by an hydride of a compound of general formula (V) in the presence of a catalyst and in the presence of a compound of general formula (VIII) to produce a compound of general formula (Ia), at a temperature of from 0° C. to 150° C. and under a pressure of from 1 bar and 100 bar.

12. A compound of general formula (II) 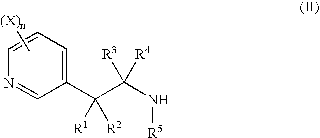
in which
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino-C₁-C₆-alkyl group, a C₁-C₈-alkyl, a C₂-C₈-alkenyl, a C₂-C₈-alkynyl, a C₁-C₆-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₂-C₈-halogenoalkenyloxy having 1 to 5 halogen atoms, a C₁-C₈-alkynyloxy, a C₃-C₈-halogenoalkynyloxy having 1 to 5 halogen atoms, a C₃-C₈-cycloalkyl, a C₃-C₈ halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a (N—C₁-C₈-alkyl)oxycarbamoyl, a C₁-C₈-alkoxycarbamoyl, a (N—C₁-C₈-alkyl)-C₁-C₈-alkoxycarbamoyl, a C₁-C₈-alkoxycarbonyl, a C₁-C₉-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino)-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
n is 1, 2, 3 or 4,
R¹ and R² are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₆-alkyl group, a C₁-C₆-alkyl, a C₂—-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group;
or R¹ and R² may together form a 3-, 4-, 5- or 6-membered carbocycle;
R³ and R⁴ are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl, a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group;
or R³ and R⁴ may together form a 3-, 4-, 5- or 6-membered carbocycle.
R⁵ is a hydrogen atom or a C₃-C₇-cycloalkyl.

13. A compound of general formula (V) 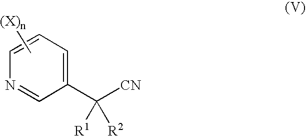
in which
X is the same or different and is a halogen atom, a nitro group, a cyano group, a hydroxy group, an amino group, a sulfanyl group, a pentafluoro-λ⁶-sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₈-alkyl, a C₂-C₈-alkenyl, a C₂-C₈-alkynyl, a C₁-C₈-alkylamino, a di-C₁-C₈-alkylamino, a C₁-C₈-alkoxy, a C₁-C₈-halogenoalkyl having 1 to 5 halogen atoms, a C₁-C₈-halogenoalkoxy having 1 to 5 halogen atoms, a C₁-C₈-alkylsulfanyl, a C₁-C₈-halogenoalkylsulfanyl having 1 to 5 halogen atoms, a C₂-C₈-alkenyloxy, a C₁-C₈-halogenoalkenyloxy having 1 to 5 halogen atoms, a C₃-C₈-alkynyloxy, a C₃-C₈-halogenoalkynyloxy having 1 to 5 halogen atoms, a C₃-C₈-cycloalkyl, a C₃-C₈-halogenocycloalkyl having 1 to 5 halogen atoms, a C₁-C₈-alkylcarbonyl, a C₁-C₈-halogenoalkylcarbonyl having 1 to 5 halogen atoms, a C₁-C₈-alkylcarbamoyl, a di-C₁-C₈-alkylcarbamoyl, a (N—C₁-C₈-alkyl)oxycarbamoyl, a C₁-C₈-alkoxycarbamoyl, a (N—C₁-C₈-alkyl)-C₁-C₈-alkoxycarbamoyl, a C₁-C₈-alkoxycarbonyl, a C₁-C₈-halogenoalkoxycarbonyl having 1 to 5 halogen atoms, a C₁-C₈-alkylcarbonyloxy, a C₁-C₈-halogenoalkylcarbonyloxy having 1 to 5 halogen atoms, a C₁-C₈-alkylcarbonylamino, a C₁-C₈-halogenoalkylcarbonylamino having 1 to 5 halogen atoms, a C₁-C₈-alkylaminocarbonyloxy, a di-C₁-C₈-alkylaminocarbonyloxy, a C₁-C₈-alkyloxycarbonyloxy, a C₁-C₈-alkylsulphenyl, a C₁-C₈-halogenoalkylsulphenyl having 1 to 5 halogen atoms, a C₁-C₈-alkylsulphinyl, a C₁-C₈-halogenoalkylsulphinyl having 1 to 5 halogen atoms, a C₁-C₈-alkylsulphonyl, a C₁-C₈-halogenoalkylsulphonyl having 1 to 5 halogen atoms, a C₁-C₆-alkoxyimino, a (C₁-C₆-alkoxyimino-C₁-C₆-alkyl, a (C₁-C₆-alkenyloxyimino)-C₁-C₆-alkyl, a (C₁-C₆-alkynyloxyimino)-C₁-C₆-alkyl, a (benzyloxyimino)-C₁-C₆-alkyl, a benzyloxy, a benzylsulfanyl, a benzylamino, a phenoxy, a phenylsulfanyl or a phenylamino;
n is 1, 2, 3 or 4;
R¹ and R² are the same or different and are a hydrogen atom, a halogen atom, a cyano group, a hydroxy group, an amino group, a sulfanyl group, a formyl group, a formyloxy group, a formylamino group, a carboxy group, a carbamoyl group, a N-hydroxycarbamoyl group, a carbamate group, a (hydroxyimino)-C₁-C₆-alkyl group, a C₁-C₆-alkyl, a C₂-C₆-alkenyl, a C₂-C₆-alkynyl, a C₁-C₆-alkylamino, a di-C₁-C₆-alkylamino, a C₁-C₆-alkoxy, a C₁-C₆-halogenoalkyl having 1 to 5 halogen atoms, a C₁-C₆-halogenoalkoxy having 1 to 5 halogen atoms, a C₁-C₆-alkylsulfanyl, a C₁-C₆-halogenoalkylsulfanyl having 1 to 5 halogen atoms, a C₂-C₆-alkenyloxy, a C₂-C₆-halogenoalkenyloxy having 1 to 5 halogen atoms, a C₃-C₆-alkynyloxy, a C₃-C₆-halogenoalkynyloxy having 1 to 5 halogen atoms, a C₃-C₆-cycloalkyl, a C₃-C₆-halogenocycloalkyl having 1 to 5 halogen atoms, a C₁-C₆-alkylcarbonyl, a C₁-C₆-halogenoalkylcarbonyl having 1 to 5 halogen atoms, a C₁-C₆-alkylcarbamoyl, a di-C₁-C₆-alkylcarbamoyl, a N—C₁-C₆-alkyloxycarbamoyl, a C₁-C₆-alkoxycarbamoyl, a N—C₁-C₆-alkyl-C₁-C₆-alkoxycarbamoyl a C₁-C₆-alkoxycarbonyl, a C₁-C₆-halogenoalkoxycarbonyl having 1 to 5 halogen atoms, a C₁-C₆-alkylcarbonyloxy, a C₁-C₆-halogenoalkylcarbonyloxy having 1 to 5 halogen atoms, a C₁-C₆-alkylcarbonylamino, a C₁-C₆-halogenoalkylcarbonylamino having 1 to 5 halogen atoms, a C₁-C₆-alkylaminocarbonyloxy, a di-C₁-C₆-alkylaminocarbonyloxy, a C₁-C₆-alkyloxycarbonyloxy, a C₁-C₆-alkylsulphenyl, a C₁-C₆-halogenoalkylsulphenyl having 1 to 5 halogen atoms, a C₁-C₆-alkylsulphinyl, a C₁-C₆-halogenoalkylsulphinyl having 1 to 5 halogen atoms, a C₁-C₆-alkylsulphonyl, a C₁-C₆-halogenoalkylsulphonyl having 1 to 5 halogen atoms, a benzyl, a benzyloxy, a benzylsulfanyl, a benzylsulfinyl, a benzylsulfonyl, a benzylamino, a phenoxy, a phenylsulfanyl, a phenylsulfinyl, a phenylsulfonyl, a phenylamino, a phenylcarbonylamino, a 2,6 dichlorophenyl-carbonylamino group or a phenyl group.
or R¹ and R² may together form a 3-, 4-, 5- or 6-membered carbocycle.

14. Fungicidal composition comprising an effective amount of a compound according to claim 1 and an agriculturally acceptable support.

15. Method for preventively or curatively combating the phytopathogenic fungi of crops, characterised in that an effective and non-phytotoxic amount of a composition according to claim 14 is applied to the plant seeds or to the plant leaves and/or to the fruits of the plants or to the soil in which the plants are growing or in which it is desired to grow them.
